(19) United States
(12) Patent Application Publication
(10) Pub. No.: US 2011/0182953 A1
(43) Pub. Date: Jul. 28, 2011

(54) POLYMORPHIC FORM OF CALCIUM ACAMPROSATE

(76) Inventors: Annibale Salvi, Milano (IT); Antonio Nardi, Segrate (IT); Bruno De Angelis, Segrate (IT)

(21) Appl. No.: 13/120,992

(22) PCT Filed: Sep. 4, 2009
(86) PCT No.: PCT/IB2009/006764

§ 371 Date: Apr. 12, 2011

(30) Foreign Application Priority Data
Sep. 26, 2009 (IT) MI2008A001706

(51) Int. Cl. A61K 9/00; C07C 309/15; A61K 31/185; A61P 25/32

(52) U.S. Cl. 424/400; 562/105; 514/578

(57) ABSTRACT

The present invention relates to a new polymorphic form of calcium acetyl-homotaurinate, designated as form B, and processes for the preparation of said form B and of the known crystalline form.

### SUMMARY OF THE INVENTION

The present invention relates to a new polymorphic form of calcium acetylhomotaurinate, the common international denomination of which is calcium acamprosate.

### TECHNICAL BACKGROUND

Calcium acamprosate (or calcium acetylhomotaurinate) which has the following formula

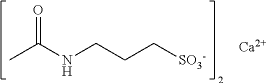

is a pharmaceutically active molecule successfully used in the treatment of alcoholism.

Preparation of calcium acamprosate has been disclosed for example in the U.S. Pat. No. 4,355,043 and its therapeutic effectiveness for the treatment of alcoholism and prevention of relapses is widely described in literature. Calcium acamprosate has the appearance of a white crystalline powder. Polymorphism is a phenomenon that affects different crystalline forms of a smile molecule. In fact, a solid crystalline phase can be organised differently in a crystalline structure, giving rise to polymorphic crystalline forms. The various crystalline forms or polymorphic forms can have different thermodynamic stability and different behaviour as regards, for example, absorption, bioavailability, solubility etc.

At the moment one single crystalline form of calcium acamprosate is known.

### DESCRIPTION OF THE INVENTION

It has now unexpectedly been found that it is possible to obtain a polymorphic form of calcium acamprosate (herebelow designated as “form B”) which is thermodynamically more stable than the known crystalline form (herebelow “form A”).

In particular a new crystallisation process has been found for obtaining, as the operating conditions used vary, the known crystalline form (form A) and the new polymorphic form B. Said polymorphic form B has been characterised by X-ray and by means of infrared (IR) spectroscopy.

Thus according to one of its aspects, the invention concerns a new polymorphic form of calcium acamprosate, designated as form B, which shows an X-ray diffraction pattern (instrument: automatic diffractometer θ/θ ITAL-STRUCTURE, copper tube with emission λ=1.54184 Å filtered by nickel plate) having the following angular positions of the most characterising peaks:
(2Th±0.2): 9.8; 11.7; 12.2; 17.7; 19.6; 20.8; 23.4; 23.7; 24.6; 20.0; 28.5; 29.7

The invention furthermore concerns the new polymorphic form B of calcium acamprosate which shows the X-ray diffraction spectrum illustrated in FIG. 1.

The invention furthermore concerns a new polymorphic form B of calcium acamprosate which shows an IR spectrum (instrument: PERKIN ELMER Spectrum 100; pure sample measured with UATR system) with characteristic absorptions approximately at the following wavelengths:
2800-3300 cm⁻¹: stretch N—H and C—H
1633 cm⁻¹: stretch C═O
1186 and 1062 cm⁻¹: stretch —SO₂O—

The invention furthermore concerns the new polymorphic form B of calcium acamprosate which shows the IR spectrum illustrated in FIG. 2.

The polymorphic form B of the invention has a greater thermodynamic stability than the known crystalline form (form A) and therefore offers many advantages with respect to the known form A.

By accelerated stability tests it was ascertained that the new polymorphic form B does not alter even when exposed to heat and/or humidity sources and is therefore more stable.

Obviously stability is an extremely important characteristic for a pharmaceutically active molecule, as is the case here.

### BRIEF DESCRIPTION OF THE DRAWINGS

FIG. 1 shows the X-ray diffraction spectrum of the new polymorphic form B of calcium acamprosate.

FIG. 2 shows the IR spectrum of the new polymorphic form B of calcium acamprosate.

FIG. 3 shows a comparison between the IR spectrum of the known crystalline form (form A) of calcium acamprosate obtained according to the present invention and the IR spectrum of calcium acamprosate reported in the European Pharmacopoeia (Edition 6.0, year 2008).

According to another of its aspects, the invention concerns a process for preparation of the crystalline forms A and B.

In particular, it has surprisingly been found that by crystallising the calcium acamprosate in water and isopropyl alcohol, it is possible to obtain the two polymorphic crystalline forms separately, by varying the temperature and reaction time conditions.

Thus, according to another of its aspects, the invention concerns a process for preparation of the polymorphic form B of calcium acamprosate which comprises the following steps:
(a) dissolving the calcium acamprosate in water at a temperature between 60° C. and the boiling temperature of the solution;
(b) adding isopropyl alcohol slowly to the solution obtained in (a), kept at a temperature between 60° C. and the boiling temperature;
(c) stirring the mixture at a temperature between 60° C. and the boiling temperature of the mixture;
(d) filtering the precipitate obtained.

According to a preferred embodiment of the invention, the temperature of steps (a), (b) and (c) is between 65 and 90° C., advantageously between 70 and 85° C., for example 75-80° C.

The quantity of water used in step (a) is preferably between 1 and 5 litres per Kg of calcium acamprosate, advantageously around 1.1-1.5 litres per Kg. The quantity of isopropyl alcohol added in step (b) is preferably between 1 and 10 litres per Kg of calcium acamprosate, advantageously around 4-5 litres per Kg. The isopropyl alcohol should be added very slowly.

According to a preferred embodiment of the invention, in step (c) the mixture is stirred for a period of longer than 10 hours, advantageously longer than 20 hours, for example for a period of between 10 and 70 hours, normally a period of 20-40 hours is sufficient to permit formation and precipitation of the desired product.

Filtering of step (d) should be performed hot, preferably at a temperature higher than 60° C.

At the end of step (d) the polymorphic form B of calcium acamprosate as designated above is obtained.

If desired or necessary, it is possible to further purify the precipitate obtained in step (d) by washing it with a mixture of isopropanol and water, for example with a 75/25 mixture of isopropanol and water, said mixture having been advantageously pre-heated to approximately 70-80° C.

The process described above allows the polymorphic form B of calcium acamprosate to be obtained which shows the X-ray diffraction characteristics and IR absorptions defined above, which are different from those reported for the known crystalline form of calcium acamprosate.

As stated above, it has surprisingly been found that by modifying the crystallisation conditions reported above, it is possible to obtain the known crystalline form of calcium acamprosate (form A).

According to another of its aspects, the invention concerns a process for the preparation of the crystalline form A of calcium acamprosate which comprises the following steps:
(e) dissolving the calcium acamprosate in water at a temperature below 55° C.;
(f) cooling the solution obtained in (e) to a temperature below 30° C. and adding isopropyl alcohol obtaining precipitation of the product;
(g) stirring the mixture at a temperature below 30° C.;
(h) filtering the precipitate obtained.

According to a preferred embodiment of the invention, the temperature of step (e) is between 35 and 50° C., for example 40-50° C.

According to another preferred embodiment, at the end of phase (e) approximately ⅓ of the water used is removed by distillation under reduced pressure. This operation increases the yield of the end product.

According to a preferred embodiment of the invention, the phases (f) and (g) are performed at room temperature, i.e. around 20-25° C.

The quantities of water and isopropyl alcohol used in steps (e) and (f) are substantially analogous to those used in steps (a) and (b) reported above for preparation of the polymorph B.

In step (g) the mixture is stirred for a short time, normally a period of 10-40 minutes up to a few hours is sufficient for complete precipitation of the desired product.

At the end of step (h) the crystalline form A of calcium acamprosate is obtained, i.e. the form known in the art.

If desired or necessary, it is possible to further purify the precipitate obtained in step (h) by washing it with isopropanol at room temperature.

The process described above allows the crystalline form A of calcium acamprosate, known in the art, to be obtained.

Further details of the processes described above are provided in the experimental section of the present description.

According to another of its aspects, the invention concerns a pharmaceutical composition comprising, as the active ingredient, the polymorphic form B of calcium acamprosate, optionally in combination with one or more pharmaceutically acceptable excipients.

The pharmaceutical composition of the invention can be formulated according to the methods well known in the art, for example it can be formulated in unit dosage forms.

The appropriate administration unit forms comprise oral forms such as tablets, capsules, powders, granules and oral solutions or suspensions, sublingual administration and administration by mouth, aerosols, topical forms of administration, implants, subcutaneous, intramuscular, intravenous, intranasal or intraocular forms of administration and rectal forms of administration.

The preferred pharmaceutical forms are the oral pharmaceutical forms such as tablets or capsules, with immediate release or controlled release over time.

A preferred pharmaceutical form is a gastro-resistant form, advantageously a gastro-resistant form which comprises 333 mg of the polymorphic form B of calcium acamprosate and one or more excipients chosen from crospovidone, microcrystalline cellulose, magnesium silicate, sodium starch glycolate, colloidal silica, magnesium stearate, talc, propylene glycol and Eudragit® L 30D or other excipients with similar action.

According to another of its aspects, the invention concerns use of the polymorphic form B of calcium acamprosate for the preparation of a medicament for the treatment of alcoholism, alcohol addiction and for preventing relapses in subjects suffering from alcohol withdrawal symptoms. The polymorphic form B of calcium acamprosate as a medicament for the treatment of alcoholism, alcohol addiction and for preventing relapses in subjects suffering from alcohol withdrawal symptoms constitutes a further subject of the invention.

The following examples illustrate the invention in a non-limiting manner.

### EXPERIMENTAL SECTION

### Example 1

### Preparation of Form B of Calcium Acamprosate

110 g (275 mmoles) of calcium acamprosate are added to 150 ml of deionised water. The mixture is heated to 75° C. and stirred for a few minutes obtaining complete solution. Maintaining the internal temperature at 75-80° C., 500 ml of isopropyl alcohol are added dropwise, taking approximately 5 hours, obtaining precipitation of the product. The mixture is stirred at 75-80° C. for 48 hours. The white solid is filtered hot (75-80° C.) and washed with 300 ml of a 75/25 mixture of isopropyl alcohol and deionised water, pre-heated to 70° C. The wet product is dried at 45° C. under reduced pressure for 6 hours. 44 g of calcium acamprosate are obtained consisting entirely of a new crystalline form (form B).

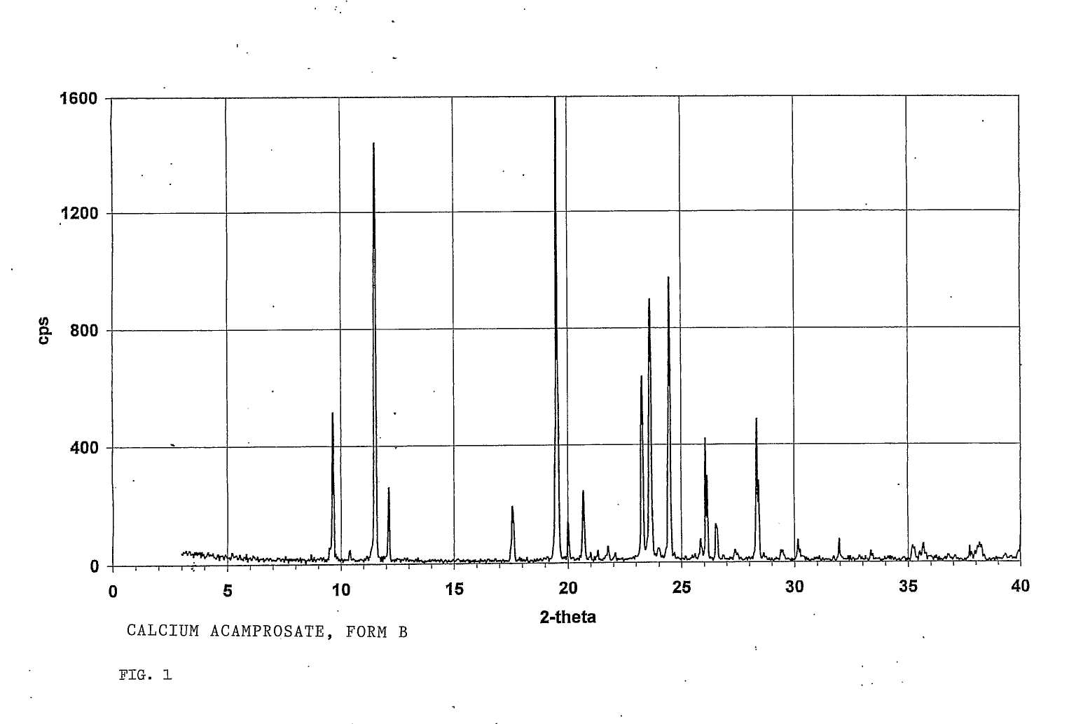

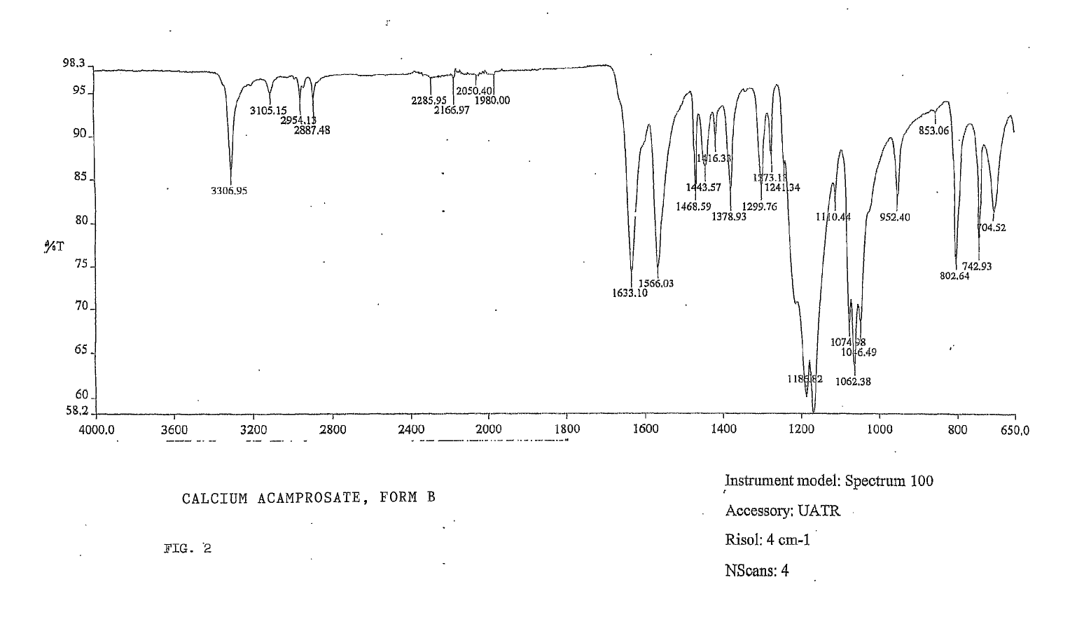

The characterisation of the new crystalline form was characterised by means of XRD (instrument: automatic diffractometer θ/θ ITAL-STRUCTURE, copper tube with emission λ=1.54184 Å filtered by nickel plate) and FT-IR spectroscopy (instrument PERKIN ELMER Spectrum 100; pure sample measured with UATR system) the results of which are reported in FIGS. 1 and 2.

### Example 2

### Preparation of Form A of Calcium Acamprosate

22 g (55 mmoles) of calcium acamprosate are added to 32 ml of deionised water. It is heated to 40-50° C. and stirred for a few minutes obtaining complete solution. 10-12 ml of water are distilled under reduced pressure and at an internal temperature of 40-50° C. The solution thus concentrated is cooled to 20-25° C. and 94 ml of isopropyl alcohol are added dropwise, taking 15-20 minutes, resulting in precipitation of the product. It is stirred at 20-25° C. for 30 minutes. The white solid is filtered and washed with 30 ml of isopropyl alcohol. The wet product is dried at 45° C. under reduced pressure for 6 hours. 18.9 g of calcium acamprosate are obtained consisting entirely of the known crystalline form (form A).

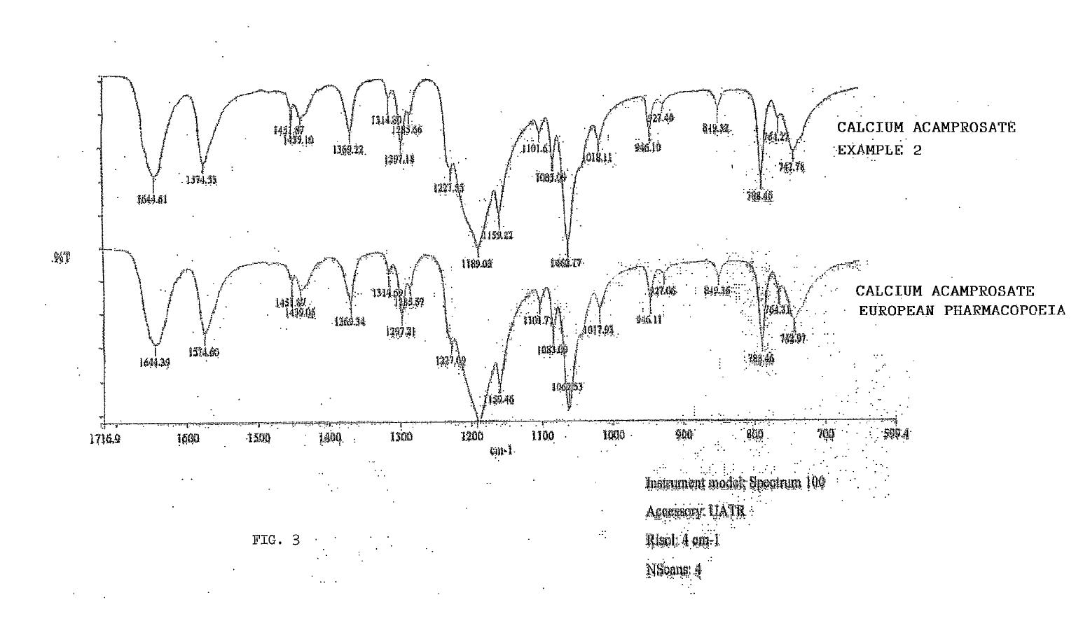

In particular the crystalline form was allocated following comparison of the FT-IR spectrum of the compound obtained from example 2 with the FT-IR spectrum reported as reference spectrum in the “EUROPEAN PHARMACOPOEIA” (Edition 6.0, year 2008). The comparison highlights a substantial overlap of the two spectra as shown in FIG. 3.

1. New polymorphic form of calcium acamprosate, designated as form B, showing an X-ray diffraction pattern having the following angular positions of the most characterizing peaks:
(2Th±0.2): 9.8; 11.7; 12.2; 17.7; 19.6; 20.8; 23.4; 23.7; 211.6; 20.0; 28.5; 29.7

2. New polymorphic form of calcium acamprosate, designated as form B, showing the X-ray diffraction pattern of FIG. 1.

3. New polymorphic form of calcium acamprosate, designated as form B, showing an IR spectrum having characteristic absorptions at approximately the following wavelengths:
2800-3300 cm⁻¹: stretch N—H e C—H;
1633 cm⁻¹: stretch C═O;
1186 and 1062 cm⁻¹: stretch —SO₂O—.

4. New polymorphic form of calcium acamprosate, designated as form B, showing the IR spectrum of FIG. 2.

5. A process for the preparation of the polymorphic form B of calcium acamprosate according to claim 1, which comprises the following steps:
(a) dissolving calcium acamprosate in water at a temperature comprised between 60° C. and the boiling point of the solution;
(b) slowly adding isopropyl alcohol to the solution obtained in (a), kept at a temperature comprised between 60° C. and the boiling point of the mixture;
(c) stirring the mixture at a temperature comprised between 60° C. and the boiling point of the mixture;
(d) filtering the precipitate thus obtained.

6. The process according to claim 5, characterized in that the temperature of steps (a), (b) and (c) is comprised between 70 and 85° C.

7. A process for the preparation of the polymorphic form A of calcium acamprosate, which comprises the following steps:
(e) dissolving calcium acamprosate in water at a temperature lower than 55° C.;
(f) cooling the solution obtained in (e) at a temperature lower than 30° C. and adding isopropyl alcohol;
(g) stirring the mixture at a temperature lower 30° C.;
(h) filtering the precipitate thus obtained.

8. The process according to claim 7, characterized in that the temperature of step (e) is comprised between 20 and 25° C.

9. A pharmaceutical composition comprising, as the active ingredient, the polymorphic form B of calcium acamprosate according to anyone of claim 1, optionally in combination with one or more pharmaceutically acceptable carriers.

10. The pharmaceutical composition according to claim 9 which is in the form of an enteric coated composition.

11. Use of the polymorphic form B of calcium acamprosate according to claim 1, for the preparation of a medicament for the treatment of alcoholism, of alcohol addiction and for preventing relapses in subjects suffering of alcohol withdrawal symptoms.

12. Polymorphic form B of calcium acamprosate according to claim 1, for its use as a medicament for the treatment of alcoholism, of alcohol addiction and for preventing relapses in subjects suffering of alcohol withdrawal symptoms.
